(19) United States
(12) Patent Application Publication
(10) Pub. No.: US 2008/0009893 A1
(43) Pub. Date: Jan. 10, 2008

(54) Lancing Device with Releasable Threaded Enclosure

(75) Inventor: Richard W. LeVaughn, Newnan, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: FACET TECHNOLOGIES, LLC, Kennesaw, GA (US)

(21) Appl. No.: 11/722,055

(22) PCT Filed: Dec. 20, 2005
(86) PCT No.: PCT/US05/46117

§ 371 Date: Jun. 18, 2007

Related U.S. Application Data
(60) Provisional application No. 60/637,720, filed on Dec. 20, 2004.

(51) Int. Cl. A61B 5/151

(52) U.S. Cl. 606/181

(57) ABSTRACT

A lancing device having a body and an endcap, the body and the endcap being connected to one another by a threaded coupling. One or the other of the body and the endcap has a threaded collar with at least one slot formed therein to permit the collar to flex upon application of excess force, so that the threaded coupling momentarily releases, skips a thread, and then re-engages, to relieve the strain of over-tightening and thereby prevent damage to the threads.

### CROSS-REFERENCE TO RELATED APPLICATIONS

This application claims the benefit of U.S. Provisional Patent Application Ser. No. 60/637,720, filed Dec. 20, 2004, the entirety of which is hereby incorporated herein by reference for all purposes.

### TECHNICAL FIELD

The present invention relates generally to lancing devices for medical sampling of body fluids, and more particularly to a lancing device having a threaded endcap with a slotted collar on which the threads are carried.

### BACKGROUND OF THE INVENTION

Various types of lancing devices are used for pricking the skin of a human or animal subject to obtain a sample of blood or other bodily fluid for medical analysis, as in the case of blood glucose sampling by diabetics, blood typing, and the like. Some lancing devices include a threaded endcap to permit removal for replacement of the lancet(s), and/or to permit the user to adjust the depth of penetration of the lancet tip into the subject's skin at the sampling site. For example, U.S. Pat. No. 6,045,567 to Taylor et al, issued Apr. 4, 2000, which is incorporated herein by reference, includes a cap that is removably joined to the housing by a screw thread.

It has been found that the threading on such caps can be damaged due to misuse, wear, or exposure to chemicals, rendering the device unusable. For example, common forms of misuse that can damage cap threads include over-tightening, snapping the caps off instead of twisting them, as well as cross-threading the cap into place. And chemicals found in common substances such as hand lotions can degrade the plastic materials often used to fabricate lancing devices, weakening the threads and rendering them more prone to damage. Additionally, the threads of many endcaps are under constant tension as a result of the torque that holds them in place, which can accelerate thread damage.

Previous attempts to address this problem have included providing a snap-on cap, rather than a threaded connection. A disadvantage of this approach is that such caps are often quite sensitive to the orientation of the cap as it is installed. This limits the ease of use of the device and can be frustrating for a user with impaired vision (which is often the case for diabetics, who are frequent users of lancing devices). Snap-on connections also often require features that are difficult to mold, and the snap geometry is often very tolerance-sensitive, requiring fine-tuning of the engagement features to produce the desired manner of operation and release force, thereby increasing manufacturing difficulty and cost. And finally, a snap connection is typically less precise at holding the two parts together, resulting in a higher degree of dimensional variance on the assembled device. Because the positioning of the endcap is a common mechanism of controlling the depth of lancet puncture by a lancing device, inconsistent dimensional control of the cap connection will therefore result in variation in the depth of puncture. This is generally undesirable, as too shallow a penetration depth will not produce an adequate sample size, necessitating multiple lancing operations; and too deep a penetration depth can result in unnecessary pain for the subject.

Thus it can be seen that needs exist for an improved lancing device and connection means for attaching an endcap to the body of the lancing device. It is to the provision of such improvements meeting these and other needs that the present invention is primarily directed.

### SUMMARY OF THE INVENTION

The present invention provides an improved lancing device and an improved endcap for a lancing device, preferably having a threaded cap connection that is less susceptible to damage from misuse or prolonged stress. The endcap of the present invention preferably provides increased reliability and improved performance by providing a threaded connection between the endcap and the lancing device body, wherein an automatic release mechanism protects the threads from damage due to over-tightening, cross-threading, and/or other improper removal or installation techniques.

In one aspect, the invention is an improvement to a lancing device of the type having a body and an endcap. The improvement preferably includes a threaded collar for connection of the endcap to the body, wherein the threaded collar has at least one slot therethrough.

In another aspect, the present invention is a lancing device including a body and an endcap, wherein at least one of the body and/or the endcap has a collar for connection of the endcap to the body. The collar preferably has at least one slot formed therein to permit at least a portion of the collar to flex upon application of excess force.

In still another aspect, the present invention is an endcap for a lancing device. The endcap preferably includes a distal end having a lancet opening formed therethrough, and a proximal end having a collar extending therefrom. The collar preferably has at least one slot formed therein to permit at least a portion of the collar to resiliently deflect upon application of excess force.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective assembly view of a lancing device with an endcap according to an example embodiment of the present invention.

FIG. 1a is a perspective view of the lancing device of FIG. 1, showing the endcap attachment in greater detail.

FIG. 2 shows the lancing device of FIG. 1, with the endcap being threaded onto the lancing device body.

FIG. 3a is a perspective view of a previously known threaded endcap.

FIG. 3b is a detailed perspective view of a threaded endcap according to an example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

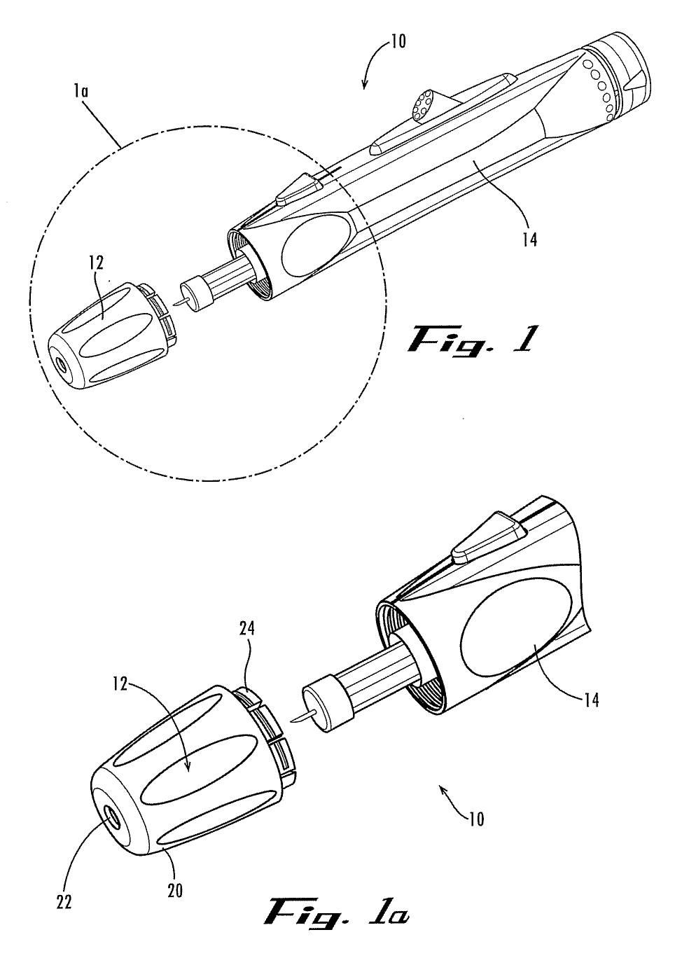

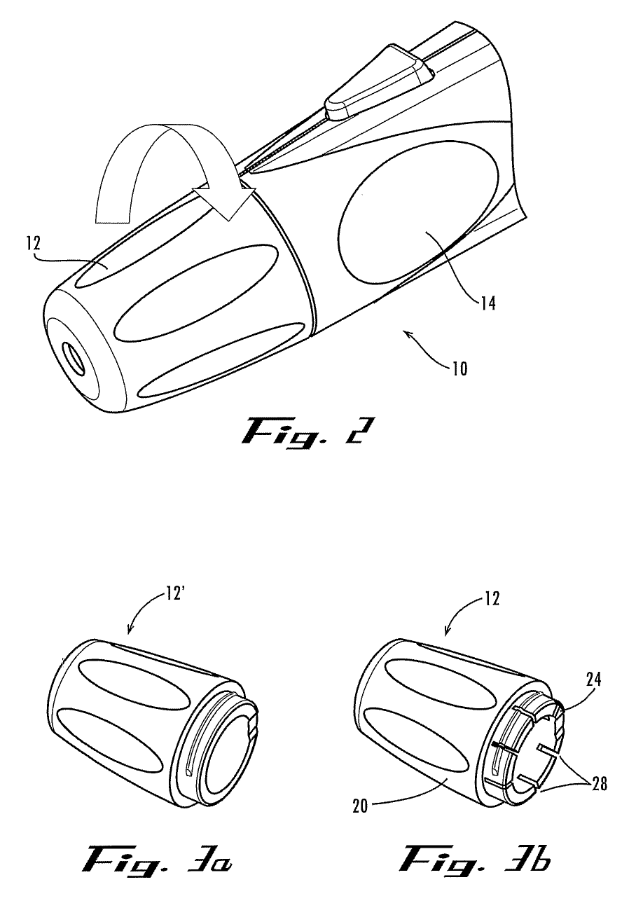

With reference now to the drawing figures, a lancing device 10 according to an example form of the present invention is shown. The lancing device 10 preferably has an endcap 12 connected to the body 14 of the lancing device by a threaded connection, shown in greater detail in FIGS. 1a and 3b.

The lancing device 10 generally comprises a drive mechanism comprising one or more springs, cams, solenoids or other drive means; and a lancet having a sharp tip, which is driven between a retracted position wherein the tip is shielded within the housing and an extended position wherein the tip projects outwardly of the housing, typically through a lancet opening in the endcap 12, to lance the subject's skin at the desired lancing site. The lancing device typically also includes a charging mechanism for energizing the drive means, and a release mechanism for actuating the device to propel the lancet along its path of travel between the retracted and extended positions. The lancing device optionally also includes a depth-control mechanism for adjusting the depth of penetration of the lancet tip into the tissue at the lancing site.

FIG. 3a shows a threaded endcap 12′ for a lancing device according to a previously known design, for example as described in U.S. Pat. No. 6,045,567, which is incorporated herein by reference. Although described and shown herein with particular reference to an improvement to a lancing device similar to that of the '567 patent, the present invention is adaptable for use in connection with any of a wide variety of lancing devices incorporating removable and/or adjustable endcap configurations.

The improved endcap 12 of the present invention is shown according to an example form in FIGS. 1 and 3b. In this example embodiment, the cap 12 comprises a generally cylindrical or semi-conical body portion 20, having a distal end comprising a lancet opening 22 for allowing passage of the lancet tip therethrough in its extended position. The body portion 20 is preferably generally circular in cross-section, and has one or more flat gripping surfaces or ridges on its exterior surface, to facilitate gripping and twisting of the cap by the user. In alternate embodiments, the body portion comprises any of a variety of configurations, including square, rectangular, triangular, polygonal, oval, or other cross-sectional forms; can be tapered, straight or arcuate along its length; and can have a smooth, textured, ridged, knurled, or otherwise configured external surface.

An internally or externally (external threads are depicted) threaded collar 24 preferably extends from a proximal end of the body portion of the endcap. The threads of the collar 24 preferably cooperatively engage complementary threads formed in or on the confronting end of the body 14 of the lancing device. In the depicted embodiment, the collar 24 is generally annular, coaxial with the sidewall of the endcap 12, and is recessed inwardly therefrom, to enable the provision of a smooth and continuous external contour at the transition between the endcap and the body 14 of the lancing device when assembled (as shown in FIG. 2). In alternate embodiments, the collar is eccentric relative to the sidewall of the endcap, and/or forms a continuous extension with the external surface of the body portion 20 of the endcap.

The collar 24 of the depicted embodiment includes one or more raised (male) helical threads on its external circumferential surface, for cooperative engagement with recessed (female) helical threads formed on the internal circumferential surface of the proximal end of the body 14 of the lancing device. In alternate embodiments, recessed (female) threads are formed in the collar, and/or the threads are provided in or on an internal circumferential face of the collar, for engagement with cooperative threads of the body 14 of the lancing device. The present invention also includes embodiments having the reverse configuration relative to that of the depicted embodiment, wherein the threaded collar extends from the body 14 of the lancing device, for engagement with cooperating threads in or on the endcap 12.

The collar 24 comprises one or more slots 28 spaced about its circumference. Preferably, a plurality of slots 28 are spaced about the collar 24, dividing the collar into two or more annular segments or fins. In the depicted embodiment, six slot(s) 28 extend through the entire thickness of the collar, and along substantially the entire length of the collar, and are relatively thin in their circumferential dimension, defining six arcuate segments or fins between adjacent slots. In alternate embodiments, the slots are wider, resulting in a more discontinuous collar configuration, whereby adjacent segments or fins are spaced further from one another and may or may not be arcuate in form. Although the slots 28 are oriented in a generally axial direction in the depicted embodiment, alternate forms of the invention include angularly offset slots. One or more threads are preferably formed on the internal and/or external faces of the segments or fins forming the collar 24, the thread(s) of adjacent segments or fins of the collar 24 being generally aligned across the intervening slot 28, for cooperative engagement with threads of the body 14 of the lancing device.

The provision of one or more slots 28 allows the threaded collar 24 to flex somewhat if excessive force is put on the threads, as by over-tightening of the endcap, causing the threaded connection between the endcap and the body of the lancing device to momentarily release and then re-seat in a more relaxed position (i.e., to skip a thread), without causing any significant damage to the threads of either the endcap 12 or the lancing device body 14. For example, in the depicted embodiment, the slots 28 permit the segments or fins of the collar 24 to deflect inwardly upon over-tightening or application of lateral force to the endcap, allowing the threads of the endcap 12 to momentarily release from the threads of the body 14, and skip a thread to release excess force without causing thread damage. Preferably, the thread(s) of the collar 24 and/or of the lancing device body 14 have forward and/or rearward thread faces that are angularly tapered, involute, radiused, or otherwise non-square thread face geometries, whereby interaction of the thread faces of the endcap threads with the thread faces of the body threads functions as a cam to assist in deflecting the segments or fins of the collar 24 inwardly or outwardly upon application of excess force, to momentarily release the threaded connection.

At least the collar portion 24 of the endcap 12 is preferably formed of a material having sufficient internal resilience to allow at least a portion of the collar to flex upon application of a force exceeding a threshold amount, and then return to its original position upon removal of the force, without incurring significant damage, permanent deflection, or fatigue. This prevents excessive shear force from being applied to the thread itself, reducing the likelihood of damage to the threaded surface. When too much torque or otherwise excessive force is applied to the cap, one or both of the elements of the threaded coupling will deflect and “skip” to the next thread to prevent the user from damaging the threads by over-tightening. This also allows the cap to be removed without damage by applying a laterally-directed side load to the cap, rather than twisting the cap to unscrew it.

An advantage of many example embodiments of the present invention over previously known “snap-on” endcaps is that the lancing device of the present invention maintains the conventional rotational “screwing” motion to attach, remove, and/or adjust the position of the endcap, whereas other solutions are instead pushed on linearly, usually with precise orientation and manufacturing tolerances required. This solution has the intuitive motion of a threaded coupling that is common in many applications (e.g., removing and replacing a toothpaste tube cap, removing and replacing the threaded cap on a soft drink bottle, etc.), and which users tend to be more familiar and comfortable with. Also the threaded connection of many example embodiments of the present invention allows more precise control of the relative positions of the two parts that are assembled together, resulting in better ability to adjust and control of the depth of puncture, and reduced sensitivity to tolerance control during manufacture. Example embodiments of the invention also provide a device that is more robust and convenient for the end user, and is more tolerant of user misuse than previously known designs, for example by allowing the user to snap off the cap and/or preventing or reducing the likelihood of a failure if excess force is applied to an endcap.

While the invention has been described with reference to preferred and example embodiments, it will be understood by those skilled in the art that a variety of modifications, additions and deletions are within the scope of the invention, as defined by the following claims.

What is claimed is:

1. In a lancing device of the type having a body and an endcap, the improvement comprising a threaded collar for connection of the endcap to the body, wherein the threaded collar has at least one slot therethrough.

2. The improvement to a lancing device according to claim 1, wherein the threaded collar comprises a plurality of slots therethrough, said plurality of slots dividing said threaded collar into segments.

3. The improvement to a lancing device according to claim 2, wherein at least one thread element is formed on said collar, and wherein portions of said at least one thread element are generally aligned along adjacent segments of the collar.

4. The improvement to a lancing device according to claim 3, wherein the at least one thread element comprises a non-square thread face geometry.

5. The improvement to a lancing device according to claim 2, wherein each of said segments flexes to momentarily release connection of the endcap and the body upon application of excess force, without causing thread damage.

6. The improvement to a lancing device according to claim 1, wherein the threaded collar extends from a proximal end of the endcap.

7. The improvement to a lancing device according to claim 6, wherein the threaded collar comprises an external threaded surface for engagement with a cooperating internal threaded surface of the body of the lancing device.

8. The improvement to a lancing device according to claim 7, wherein the external threaded surface has at least one male thread thereon, for engagement with a female thread formed in the internal threaded surface of the body of the lancing device.

9. A lancing device comprising a body and an endcap, at least one of the body and the endcap comprising a collar for connection of the endcap to the body, wherein the collar has at least one slot formed therein to permit flexure of at least a portion of the collar upon application of excess force.

10. The lancing device of claim 9, wherein the collar is threaded.

11. The lancing device of claim 10, wherein the threaded collar extends from a proximal end of the endcap.

12. The lancing device of claim 11, wherein the threaded collar comprises an external threaded surface for engagement with a cooperating internal threaded surface of the body of the lancing device.

13. The lancing device of claim 12, wherein the external threaded surface of the lancing device has at least one male thread thereon, for engagement with a female thread formed in the internal threaded surface of the body of the lancing device.

14. The lancing device of claim 10, wherein at least one thread element formed on the threaded collar comprises a non-square thread face geometry.

15. The lancing device of claim 9, wherein the collar comprises a plurality of slots therethrough, said plurality of slots dividing said collar into segments.

16. The lancing device of claim 15, wherein at least one thread element extends in general alignment along adjacent segments of the collar across an intermediate one of said plurality of slots.

17. The lancing device of claim 15, wherein at least a portion of each of said segments flexes to momentarily release connection of the endcap and the body upon application of excess force.

18. An endcap for a lancing device, said endcap comprising:
a distal end having a lancet opening formed therethrough; and
a proximal end having a collar extending therefrom, wherein said collar comprises at least one slot formed therein to permit at least a portion of said collar to resiliently deflect.

19. The endcap of claim 18, wherein the collar is threaded.

20. The endcap of claim 19, wherein the threaded collar comprises an externally threaded surface.

21. The endcap of claim 20, wherein the externally threaded surface has at least one male thread thereon.

22. The endcap of claim 19, wherein at least one thread element formed on the threaded collar comprises a non-square thread face geometry.

23. The endcap of claim 18, wherein the collar comprises a plurality of slots therethrough, said plurality of slots dividing said collar into segments.

24. The endcap of claim 23, wherein at least one thread element extends in general alignment along adjacent segments of the collar across an intermediate one of said plurality of slots.

25. The endcap of claim 23, wherein at least a portion of each of said segments flexes to momentarily release connection of the endcap and the body upon application of excess force.
